(19) United States
(12) Patent Application Publication
(10) Pub. No.: US 2009/0234392 A1
(43) Pub. Date: Sep. 17, 2009

(54) Method for inserting a spinal fixation element using implants having guide tabs

(75) Inventors: Sara Dziedzic, Dorchester, MA (US); Michael Mahoney, Middletown, RI (US); Christopher L. Ramsay, West Wareham, MA (US); James Roveda, N. Attleboro, MA (US)

Correspondence Address: LAHIVE & COCKFIELD, LLP;FLOOR 30, SUITE 3000, ONE POST OFFICE SQUARE, BOSTON, MA, 02109, US

(73) Assignee: DePuy Spine, Inc., Raynham, MA (US)

(21) Appl. No.: 12/075,733

(22) Filed: Mar. 13, 2008

(51) Int. Cl. A61B 17/88; A61B 17/04; A61F 2/44

(52) U.S. Cl. 606/279; 606/301; 623/17.11

(57) ABSTRACT

The present invention provides methods for inserting a spinal fixation element using minimally invasive surgical techniques. The methods use implants having guide tabs. The guide tabs are used to locate the spinal fixation element along a patient's spine. The guide tabs may be orientated in different ways to accommodate different insertion techniques for the spinal fixation element.

### FIELD OF THE INVENTION

The present invention relates to spinal fixation and methods for use during orthopedic surgery. More particularly, the present invention relates to inserting a spinal fixation element, such as a rod, using guide tabs extending from bone anchor implanted along a patient's spine.

### BACKGROUND OF THE INVENTION

Spinal fixation systems may be used in surgery to align, adjust and/or fix portions of a spinal column, i.e., vertebrae, in a desired spatial relationship relative to each other. Many spinal fixation systems employ a spinal rod for supporting the spine and for properly positioning components of the spine for various treatment purposes. Implants, such as vertebral bone anchors, comprising pins, bolts, screws, and hooks, engage the vertebrae and connect the supporting spinal rod to different vertebrae. Spinal rods can be anchored to specific portions of the vertebra. Since each vertebra varies in shape and size, a variety of anchoring devices have been developed to facilitate engagement of a particular portion of the bone.

Pedicle screw assemblies, for example, have a shape and size that is configured to engage pedicle bone. Such screws typically include a threaded shank that is adapted to be threaded into a vertebra, and a head portion having a spinal fixation element-receiving portion, which, in spinal rod applications, is usually in the form of a U-shaped slot formed in the head portion for receiving the rod. A set-screw, plug, cap or similar type of closure mechanism is used to lock the rod into the rod-receiving portion of the pedicle screw.

In conventional spinal surgery, first, anchoring devices are attached to vertebra, and then a spinal rod is aligned with the anchoring devices and secured. For example, for conventional pedicle screw assemblies, first the engagement portion of each pedicle screw is threaded into a vertebra. Once the pedicle screw assembly is properly positioned, a spinal fixation rod is connected in the rod-receiving portion of each pedicle screw head. The rod is locked into place by tightening a cap or similar type of closure mechanism to securely interconnect each pedicle screw to the fixation rod. This type of conventional spinal surgical technique usually involves making a surgical access opening in the back of the patient that is almost as long as the length of the spinal rod to be implanted. Because exact placement of the screw assemblies depends on a patient's particular bone structure and bone quality, the exact position of all screw assemblies cannot be known until after all the assemblies are positioned. Adjustments, such as bending, are made to the spinal rod to ensure that it aligns with each screw assembly.

Recently, the trend in spinal surgery has been moving toward providing minimally invasive surgical (MIS) devices and methods for implanting spinal fixation elements. In minimally invasive surgical techniques, the anchors and rod are typically inserted through small incisions. For example, the anchors and rod may be delivered percutaneously to an implant site through a small access port such as a cannula. In other methodologies, a mini-open technique may be used to place the spinal fixation system.

However, such minimally invasive procedures introduce other issues. Because the bone anchors and spinal fixation element are inserted through small incisions, such as percutaneously, there is reduced visibility of the surgical site. Placement of the spinal fixation element becomes more difficult when there is no direct view of the surgical site. Thus, what is needed is a means for being able to accurately insert a spinal fixation element along a patient's spine when using minimally invasive surgical techniques.

### SUMMARY OF THE INVENTION

Embodiments of the present invention provide methods for inserting a spinal fixation element using minimally invasive surgical techniques. The methods use implants having guide tabs. The guide tabs are used to locate the spinal fixation element along a patient's spine. The guide tabs may be orientated in different ways to accommodate different insertion techniques for the spinal fixation element.

In accordance with one aspect, a method is provided for inserting a spinal fixation element along a patient's spine, the method involves inserting a first implant having a guide tab into a first vertebra. A second implant having a guide tab may then be inserted into a second vertebra. The spinal fixation element may then be inserted using the guide tabs of the first and second implants to locate the spinal fixation element along the patient's spine.

In other embodiments, the spinal fixation element may be inserted before the first or second implant. After the implants are inserted, the spinal fixation element may be moved into position using the guide tabs to locate the spinal fixation element along the patient's spine.

In certain embodiments, the bone anchor is an anchor bolt. The anchor bolt has a bone engagement portion; a threaded head portion for receiving a locking member; and a rod connector body disposed between the bone engagement portion and the threaded head portion for connector bodying a rod. In certain embodiments the anchor bolt may include a detachable extension shaft that extends from the threaded head portion opposite the bone engagement portion.

In accordance with another aspect, a method is provided for inserting a spinal fixation element along a patient's spine. The method involves percutaneously inserting a first implant having a guide tab into a first vertebra. A second implant having a guide tab may then be percutaneously inserted into a second vertebra. The guide tabs of the first and second implants may then be orientated to define a channel along the patient's spine for receiving the spinal fixation element. The spinal fixation element may then be percutaneously inserted within the channel defined by the guide tabs of the first and second implants to locate the spinal fixation element along the patient's spine.

In accordance with another aspect, a method is provided for inserting a spinal fixation element along a patient's spine. The method involves making a mini-open incision along the patient's spine. A first implant having a guide tab may then be inserted, through the mini-open incision, into a first vertebra proximal to a first end of the mini-open incision. A second implant having a guide tab may then be inserted, through the mini-open incision, into a second vertebra proximal to a second end of the mini-open incision. The guide tabs may then be orientated to receive and guide the ends of the spinal fixation element. The spinal fixation element may then be inserted between the guide tabs, wherein the guide tabs of the first and second implants receive the first and second ends of the spinal fixation element and guide the insertion of the spinal fixation element along the patient's spine.

### BRIEF DESCRIPTION OF THE FIGURES

These and other features and advantages of the mechanisms and methods disclosed herein will be more fully understood by reference to the following detailed description in conjunction with the attached drawings in which like reference numerals refer to like elements through the different views. The drawings illustrate principles of the instruments and methods disclosed herein and, although not to scale, show relative dimensions.

FIG. 1A illustrates an exploded view of components of an implant in accordance with aspects of the present invention;

FIG. 1B illustrates an assembled view of components of an implant in accordance with aspects of the present invention;

FIG. 2 is a flow chart of one exemplary method in accordance with aspects of the present invention;

FIG. 3A is a posterior view of six percutaneous incisions formed in the thoracolumbar fascia of a patient's back;

FIG. 3B is an end view showing a blunt dissection of the muscles surrounding a patient's vertebra;

FIG. 3C is an end view of the vertebra in FIG. 3B with a k-wire placed through the incision and into the patient's vertebra;

FIG. 3D is an end view of the vertebra in FIG. 3C showing an obturator and several dilators disposed over the k-wire to dilate the tissue and muscles;

FIG. 4 illustrates an exemplary embodiment of implants inserted in accordance with aspects of the present invention;

FIG. 5A illustrates an exemplary embodiment of the insertion of the spinal fixation element in accordance with aspects of the present invention;

FIG. 5B illustrates an exemplary embodiment of a spinal fixation element inserted in accordance with aspects of the present invention;

FIG. 6A illustrates an exemplary embodiment of inserted implants in an alternate orientation in accordance with aspects of the present invention;

FIG. 6B illustrates an exemplary embodiment of the manipulation of a spinal fixation element using the guide tabs of the implants in accordance with aspects of the present invention;

FIG. 7 illustrates an exemplary embodiment of the connecting of the spinal fixation element to the implants using closing mechanism in accordance with aspects of the present invention;

FIG. 8 is a flow chart of another exemplary method in accordance with aspects of the present invention;

FIG. 9 is a flow chart of another exemplary method in accordance with aspects of the present invention

FIG. 10 illustrates an exemplary embodiment of the insertion of the implants and spinal fixation element using a mini-incision surgical technique.

### DETAILED DESCRIPTION OF THE INVENTION

Certain exemplary embodiments will now be described to provide an overall understanding of the principles of the structure, function, manufacture, and use of the adaptable clamping mechanisms and methods disclosed herein. Examples of these embodiments are illustrated in the accompanying drawings. Those of ordinary skill in the art will understand that the methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

As discussed above, embodiments of the present invention provide methods for inserting a spinal fixation element using minimally invasive surgical techniques. The implants used in exemplary embodiments have a guide tab which is used to locate the spinal fixation element along the patient's spine. Exemplary embodiments described herein concern implants for securing spinal fixation elements and methods of use. As such, exemplary embodiments of implants are formed of suitable materials for use in a human body. Suitable materials include, but are not limited to, stainless steel, titanium, or the like. Exemplary embodiments of implants are sized and dimensioned for insertion through a minimally invasive surgical access port, such as a cannula.

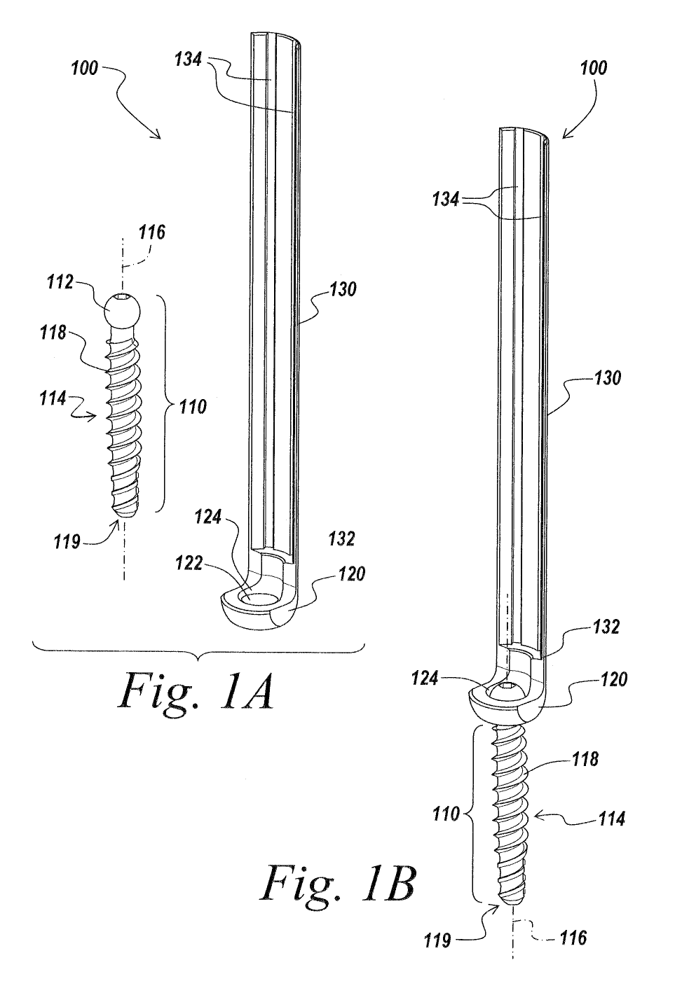

An example of one embodiment of a suitable implant can be seen in FIGS. 1A and 1B. FIG. 1A depicts a perspective view showing the individual parts of the implant 100. FIG. 1B depicts a perspective view showing the implant 100 assembled. In this example, the implant 100 includes a bone anchor 110, a connector body 120, and a detachable guide tab 130.

The bone anchor 110 comprises a joint portion, illustrated as a proximal anchor head 112, for coupling the bone anchor 110 to the connector body 120 and an anchoring portion, illustrated as a distal shaft 114 configured to engage bone. The distal shaft 114 of the bone anchor 110 extends along a longitudinal axis 116. The distal shaft 114 may include one or more bone engagement mechanisms to facilitate gripping engagement of the bone anchor to bone. In the illustrated embodiment, the distal shaft 114 includes an external thread 118 extending along at least a portion of the shaft for engaging bone. In the illustrated embodiment, the external thread 118 is a single lead thread that extends from a distal tip 119 of the shaft to the anchor head 112, though one skilled in the art will recognize that the external thread may extend along any selected portion of the shaft and have any suitable number of leads. Other suitable bone engagement mechanisms include, but are not limited to, one or more annular ridges, multiple threads, dual lead threads, variable pitched threads and/or any conventional bone engagement mechanism.

The anchor head 112 of the bone anchor 110 may be configured to facilitate adjustment of the bone anchor 110 relative to the connector body 120 of the implant 100. For example, the illustrative anchor head 112 may be substantially spherical to permit pivoting of the bone anchor 110 relative to the connector body 120 in one or more selected directions. In some embodiments, the anchor head 112 may also have surface texturing, knurling and/or ridges.

In this example, the connector body 120 forms a seat 124 for receiving a spinal fixation element. A cavity 122 passes through the connector body 120 and is configured for receiving the bone anchor 110 and engaging the proximal head 112 of the bone anchor 110.

The connector body 120 receives the proximal head 112 of the bone anchor in the cavity 122 to couple the bone anchor 110 thereto. An example of this can be seen in FIG. 1B. The connector body 120 receives a spinal fixation element in the seat 124 defined by the connector body 120, thereby coupling the spinal fixation element engaged by the connector body 120 to the bone anchor 110.

The cavity 122 of the connector body 120 is configured to interact with the spherical shape of the proximal head 112 of the bone anchor 110 to allow the bone anchor 110 to rotate and pivot independently of the connecter body 120 providing a polyaxial implant 100. Likewise, once the distal shaft 114 of the bone anchor 110 has been implanted in a bone (not shown) the interaction of the cavity 122 and proximal head 112 allow the connecter body 120 to be positioned to engage a spinal fixation element (not shown).

The detachable guide tab 130 extends from the connector body 120 in the longitudinal axis 116 opposite and offset of the distal shaft 114 of the bone anchor 110. Having the guide tab 130 offset provides reduced access to the proximal head 112 of the bone anchor 110 for inserting and potentially re-adjusting the height of the bone anchor 110. This is particularly beneficial in longer segment constructs where the bone anchor heights may need to be adjusted to match the curve of the spinal fixation element. Having one offset tab allows adjustment with the spinal fixation element not having to be removed from the site, rather just shifted lateral to the tab. Having the guide tab 130 offset also allows for side and top loading of a spinal fixation element (not shown) upon the seat 124 while still providing a guide for locating the spinal fixation element (not shown).

The detachable guide tab 130 is configured to extend outside the patient through the patient's skin while providing clear access to the connector body 120 and the proximal head 112 of the bone anchor 110. Accordingly, the guide tab 130 may form a partial cannula extending through the skin wherein the guide tab has a crescent shaped cross section. In some embodiments, additional components could be mated to the guide tab to more fully enclose the access site. In certain embodiments, the detachable guide tab 130 has a break-away feature 132 that may be fragile or weakened allowing the detachable guide tab 130 to be detached and removed. Alternatively, the detachable guide tab 130 can be detached by cutting the guide tab away from the implant 100. In still other embodiments, a secondary support could be used in conjunction with the guide tab to provide additional strength until breaking and removal is required. Other possible configurations and techniques will be apparent to one skilled in the art given the benefit of this disclosure. For example, the strength of the tab could be varied to provide enough force to maneuver the bone anchor and the spinal fixation elements together thereby potentially correcting spinal vertebral segments by adjusting their relative positions. The guide tabs may also be flexible to allow easier access of instrumentation and less crowding in the incisions. Alternatively, the guide tabs may also be a combination of stiffness. For example, the distal end could be stronger to provide the strength required for adjustments, while the proximal end exiting the skin incision could be flexible or malleable.

In certain embodiments, the detachable guide tab 130 may have one or more surface configurations 134 for engaging tools, spinal fixation elements, and/or closure mechanisms to further assist in the insertion and guidance of the tools, spinal fixation elements, and/or closure mechanisms. The surface configurations 134 may be one or more tracks on the guide tab 130. In some such embodiments, the tracks may be keyed to mate with specific tools, spinal fixation elements, and/or closure mechanisms. Other possible configurations and techniques will be apparent to one skilled in the art given the benefit of this disclosure.

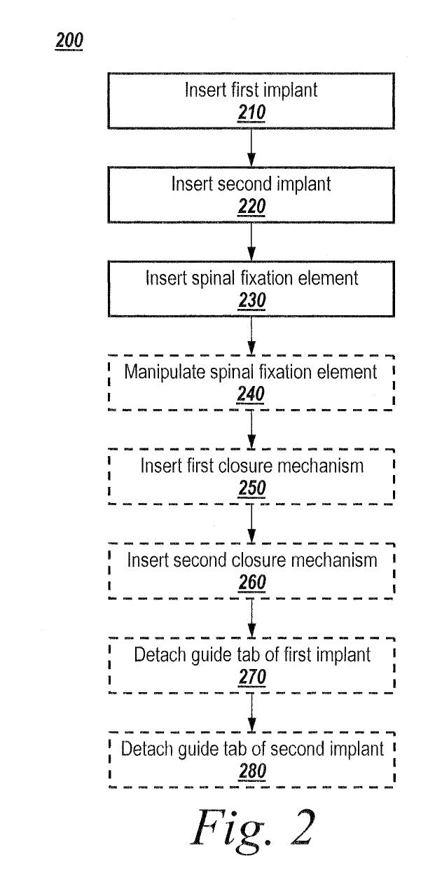

FIG. 2 depicts a flow diagram 200 of one embodiment for inserting a spinal fixation along the patient's spine in a minimally invasive surgery (MIS). The method involves using two or more implants having guide tabs, such as described above, to assist in the placement of the spinal fixation element. A first implant is inserted into a first vertebra of the patient's spine (Step 210). A second implant may then be inserted into a second vertebra of the patient's spine (Step 220). Additional implants may also be inserted as desired. A spinal fixation element, such as a rod, may then be inserted using the guide tabs of the inserted first and second implants to locate the spinal fixation element along the patients spine (Step 230). Each of these steps will be discussed in further detail below.

It should be understood that the steps set forth above are provided in one possible order, the steps can be performed in any order, for example, the spinal fixation element may be inserted into the patient before either the first and/or second implant. If the implants are inserted after the spinal fixation element, the guide tabs of the implants can still be used to locate the previously inserted spinal fixation element along the patient's spine.

In certain embodiments, the method may further include additional steps. After insertion, the spinal fixation element may be further manipulated using the guide tabs of the first and second implants (step 240). Once the implants and spinal fixation element have been inserted (and manipulated), closure mechanisms may be inserted to connect the spinal fixation element to the implants. In the example of FIG. 2, a first closure mechanism may be inserted to connect the spinal fixation element to the first implant (Step 250). A second closure mechanism may also be inserted to connect the spinal fixation element to the second implant (Step 160). Additional closure mechanism may further be inserted to connect the spinal fixation element to any additionally inserted implants as desired. Each of these steps will be discussed in further detail below.

In other embodiments, once the spinal fixation element has been connected to the implants, the guide tabs may be removed. In the example of FIG. 1, the guide tab the first implant may be detached from the first implant (Step 270) and the guide tab of the second implant may be detached from the second implant (Step 280). Additional guide tabs may further detached from any additionally inserted implants. Each of these steps will be discussed in further detail below.

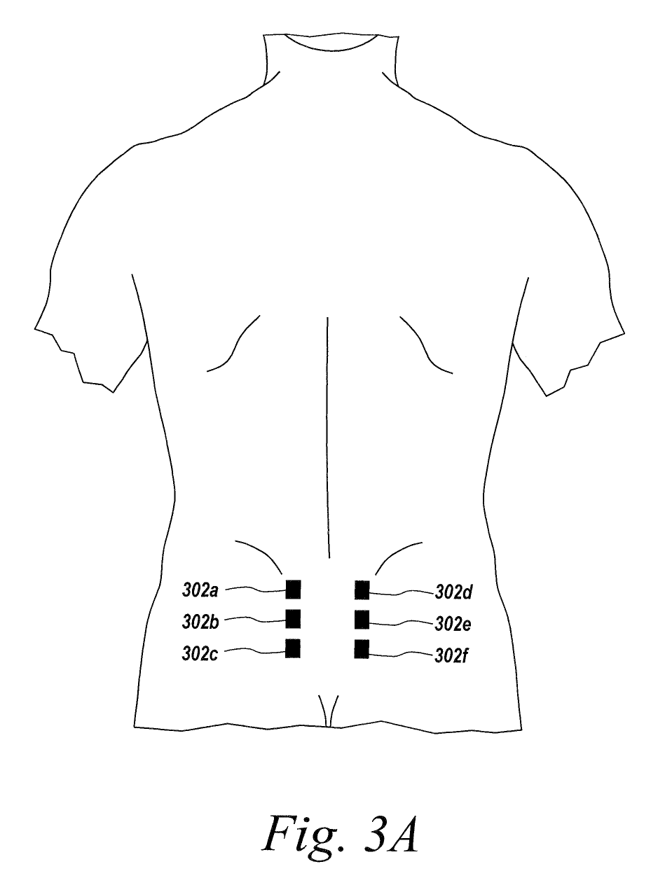

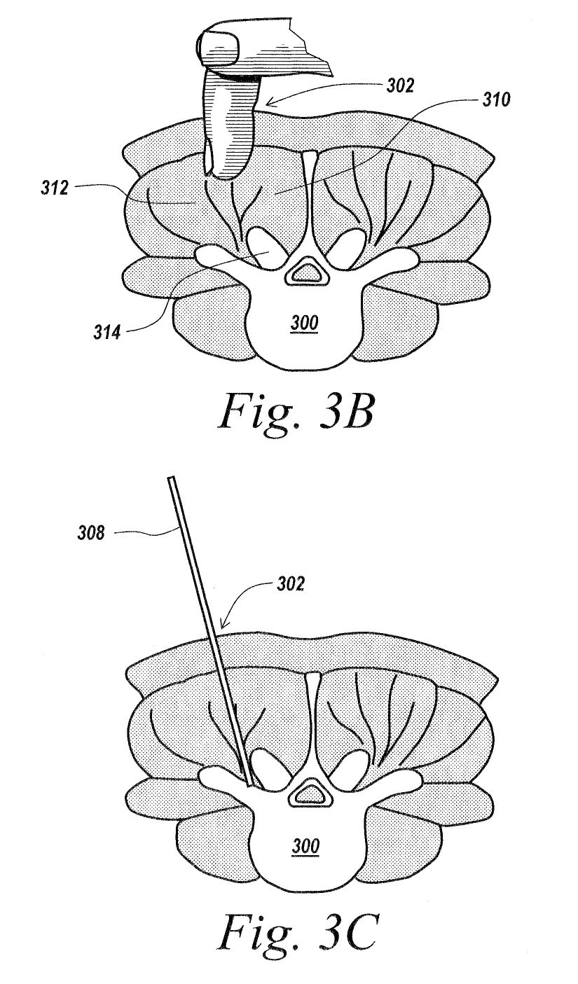

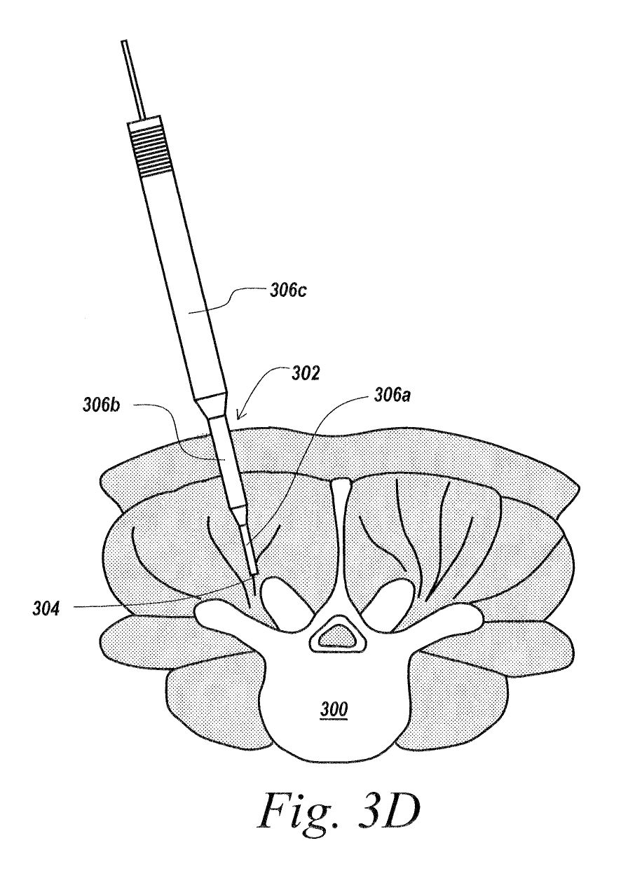

Various techniques can be used to insert the first and second implants (steps 210 and 220). For example, a minimally invasive percutaneous incision 302 may be made through the tissue at one or more sites as shown in FIGS. 3A-D. The location, shape, and size of the incision 302 will depend on the type and quantity of rod anchor systems being implanted, as well as the technique being employed to implant the rod anchor systems. By way of non-limiting example, FIG. 3A illustrates three minimally invasive percutaneous incisions 302a-c formed on one side of three adjacent vertebra in the thoracolumbar fascia in the patient's back, and three additional minimally invasive percutaneous incisions 302d-f formed on the opposite side of the three adjacent vertebra in the thoracolumbar fascia in the patient's back.

In certain exemplary embodiments, one or more of the incisions may be expanded to create a pathway from the incision 302 to proximate a vertebra 300. For example, the incision 302 may be expanded by serial dilation, with a retractor such as an expandable retractor, or by any other conventional techniques. In one exemplary embodiment, blunt finger dissection can be used, as shown in FIG. 3B, to separate the longissimus thoracis 310 and multifidus muscles 312, thereby exposing the facet and the junction of the transverse process and superior articular process 314.

An implant may be inserted through one or more of the incisions and the pathways to proximate the vertebra 300. Any technique for inserting an implant can be used. In one embodiment, for example, an implant can be inserted over a guidewire, such as a k-wire. As shown in FIG. 3C, a guide wire, e.g., a k-wire 304, can be inserted, either prior to or after formation of the incision 302, at each implant site. The k-wire 304 may extend into the vertebra 300 at the desired entry point of the implant. In certain exemplary embodiments, the k-wire may be advanced into the vertebra 300. In other exemplary embodiments, the k-wire may be positioned proximate to or against the vertebra 300. Fluoroscopy or other imaging may be used to facilitate proper placement of the k-wire 304. The incision 302 may be dilated to provide a pathway for delivery of an implant to each site, in the manner discussed above, before or after placement of the guidewire. For example, FIG. 3D illustrates serial dilation at one end of the incision 302 using an obturator 306a having several dilators 306b, 306c of increasing size placed there over. The dilators 306b, 306c are delivered over the obturator 306a and k-wire 304 to essentially stretch the skin around the incision 302 and to expand the pathway to the implant site. One skilled in the art will appreciate that an implant may be advanced to a vertebra through the incision without the need for a guidewire.

Once the incision 302 is dilated to the proper size, if necessary, the vertebra 300 may be prepared using one or more bone preparation instruments, such as drills, taps, awls, burrs, probes, etc. In certain exemplary embodiments, one or more cannulae can be used to provide a pathway from the incision 302 to the anchor site for insertion of the bone preparation instruments and/or the anchor. In an exemplary embodiment, a relatively small cannula (not shown) may be used to introduce bone preparation instruments into the surgical site. Once the vertebra 300 is prepared, an implant can be delivered along the k-wire, either through the cannula, or after the cannula is removed, and implanted in the vertebra 300. Alternatively, in embodiments not employing a guidewire, the implant may be advanced through the incision, e.g., through a cannula, to the vertebra 300. A cannula, retractor, or other instrument may be employed to guide the implant to the vertebra 800.

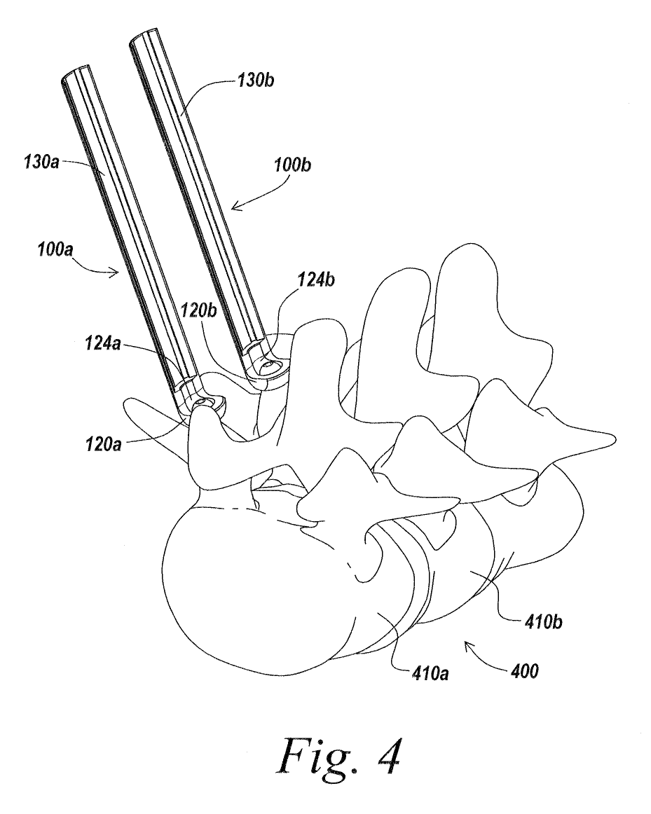

The implants may be oriented in a number of ways to assist in the insertion and targeting of a spinal fixation element. FIG. 4 depicts one such orientation wherein the first implant 100a and the second implant 100b have inserted into a first vertebra 410a and a second vertebra 410b of the patients spine 400. In this example, the bone anchors of the first implant 100a and second implant 100b have been inserted into the first 410a and second vertebra 410b and the connector bodies 120a and 120b defining the seats 124a and 124b have been orientated so that the guide tabs 130a and 130b will be on the same side of a spinal fixation element (not shown) placed on the seats 124a and 124b. This orientation allows for top and side loading of a spinal fixation element onto the seats 124a and 124b.

A variety of techniques can be used to insert a spinal fixation element, such as a rod, to extend along a patient's spine (step 230). The spinal fixation element may also be introduced at various locations along the patient's spine. For example, the spinal fixation element can be introduced through the same incision used to introduce an implant, or alternatively the spinal fixation element can be introduced through an incision that is separate from and located a distance apart from the incisions used to insert the implants. The spinal fixation element may be inserted before or after the implants. The spinal fixation element can also either be directly introduced through the incision to extend up along the patient's spinal column, or it can be introduced through a cannula, access port, or along the guide tab 130 of an implant 100 for guiding the spinal fixation element to extend along the patient's spinal column. Various tools can also be coupled to the spinal fixation element to manipulate and facilitate introduction and positioning of the spinal fixation element in the patient's body. The techniques for the percutaneous insertion of a spinal fixation element are similar to those for inserting implants as discussed above.

In one exemplary embodiment, the guide tabs 130a and 130b of the first and second implants 100a and 100b may be used to guide the spinal fixation element into location along the patient's spine. As discussed above, the guide tabs 130a and 130b of the first and second implants 100a and 100b form partial cannulae through the patient's skin to the implant site. Accordingly, the insertion of the first and second implants 100a and 100b serve to provide the incisions used to insert the first and second implants 100a and 100b with an access port, via the partial cannulae formed by the guide tabs 130a and 130b, for the insertion of a spinal fixation element.

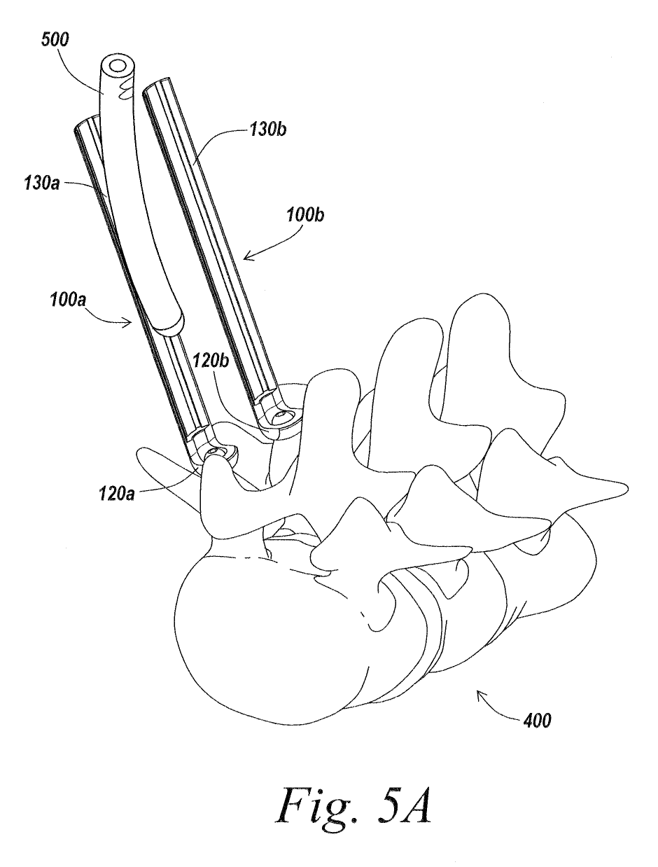

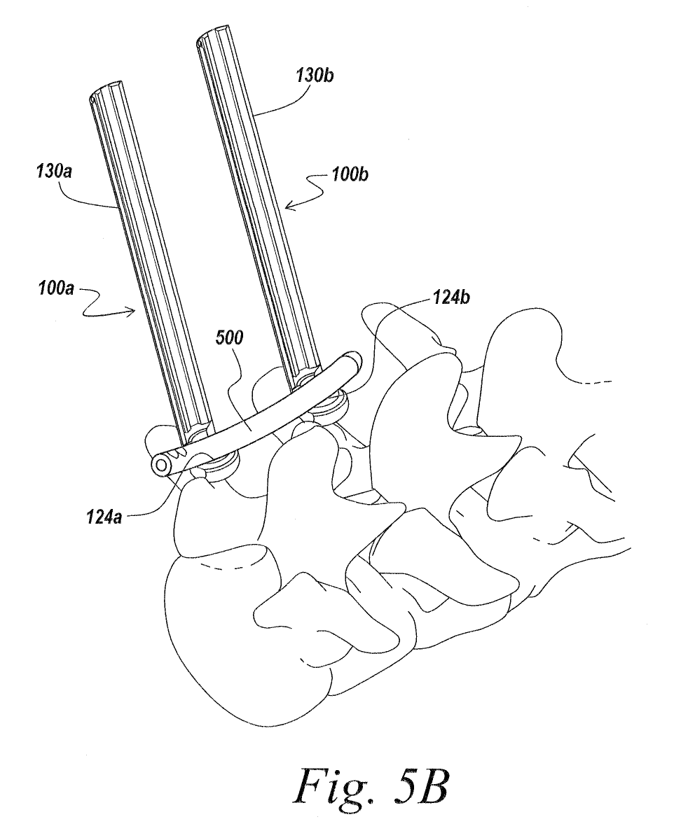

FIG. 5A depicts an exemplary embodiment wherein a spinal fixation element 500 is introduced through the same incision as used for the first implant 100a using the guide tab 130 of the first implant to direct the spinal fixation element to the target site. In this example, the spinal fixation element 500 is a rod. One end of the rod has a tapered tip 510 to aid in the insertion of the rod 500. The rod also possesses a curve along the length of the rod 500 to assist in the insertion and transition of the rod 500 from a substantially vertical orientation along the guide tab 130a to a substantially horizontal orientation along the patient's spine 400. An example of the rod 500 in a substantially horizontal orientation along a patient's spine can be seen in FIG. 5B.

It should be noted that the spinal fixation element 500 need not be introduce though one of the incisions used to insert an implant 100. The spinal fixation element 500 may be introduced through a separate incision. In certain embodiments, the spinal fixation element 500 may be inserted remotely and then the first implant 100a and second implant 100b may be inserted. Alternatively, the first implant 100a and the second implant 100b may be inserted and then the spinal fixation element 500 may be inserted.

Once the first 100a and second 100b implant have been inserted, the spinal fixation element 500 may then be positioned onto the seats 124a and 124b of the first 100a and second 100b implants. In the example of FIG. 5B, the guide tabs 130a and 130b are orientated to allow the spinal fixation element 500 to be side loaded onto the seats 124a and 124b opposite the guide tabs 130a and 130b. Other orientations of the guide tabs are also possible to assist in the insertion and location of the spinal fixation element 500.

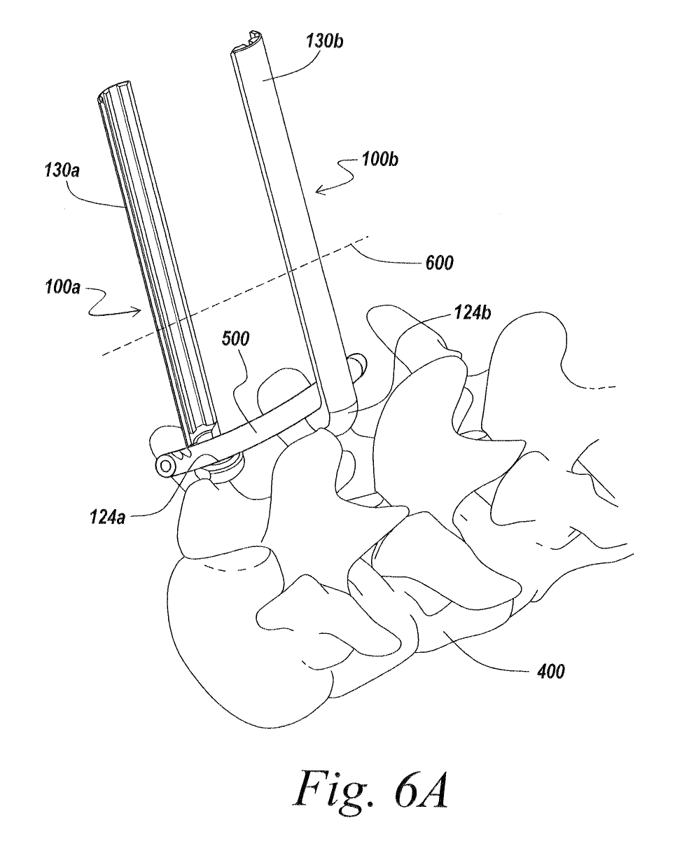

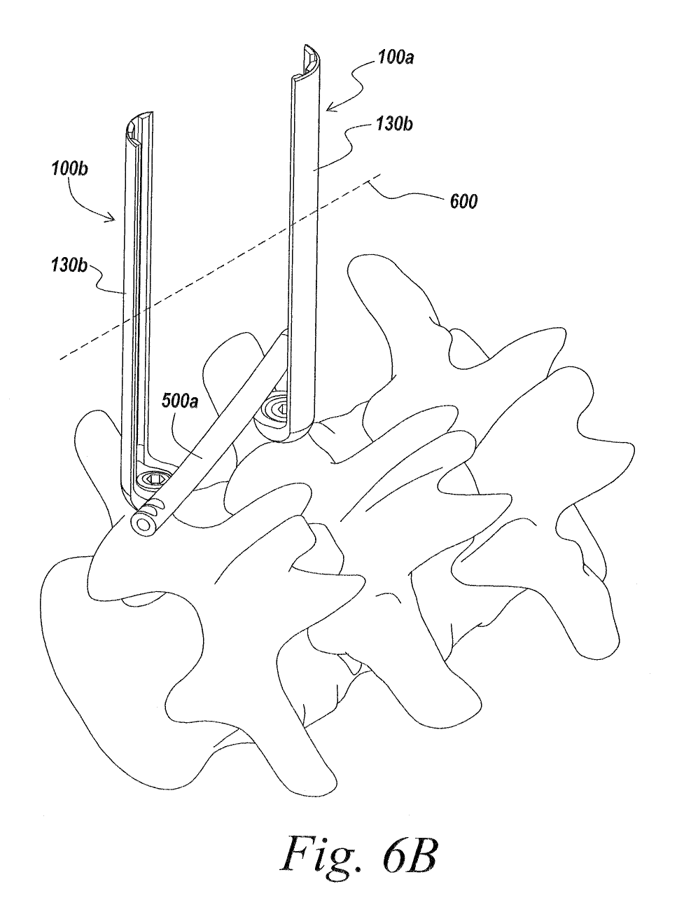

FIG. 6A depicts one embodiment of an alternate orientation of guide tabs 130a and 130b used in the insertion of a spinal fixation element 500. In this example, the guide tabs 130a and 130b are orientated so as to be on opposite sides of an inserted spinal fixation element 500. Thus, in effect, the guide tabs 130a and 130b define a channel 600 along the patient's spine 400. Inserting the spinal fixation element 500 in the channel 600 defined by the first guide tab 130a and second guide tab 130b ensures that the spinal fixation element will be positioned on the seats 124a and 124b of the first 100a and second 100b implants and located along the patient's spine 400.

In certain embodiments, once a spinal fixation element 500 has been inserted, further adjustments may be required. As such, the guide tabs 130a and 130b may be used to manipulate the position of the spinal fixation device 500 (step 240). An example of this can be seen in FIG. 6B.

As discussed above, the implants 100a and 100b are polyaxial in nature wherein the connector bodies 120a and 120b are pivotable around the proximal heads 112a and 112b of the bone anchors 110a and 110b. As such, the connector bodies 120a and 120b may be pivoted around the proximal heads 112a and 112b to adjust the guide tabs 130a and 130b to different orientations as desired. As the guide tabs 130a and 130b extend outside the patient, the guide tabs 130a and 130b provide a convenient means for manipulating the position of the connector bodies 120a and 120b to which the guide tabs 130a and 130b are attached. In the example of FIG. 6B, since the guide tabs 130a and 130b are orientated to be on both sides of the spinal fixation element, the guide tabs 130a and 130b also provide a means to manipulate the spinal fixation element 500. In this embodiment, the guide tabs 130a and 130b may be rotated, pivoted, or otherwise moved by the surgeon to come in contact with the spinal fixation element 500 and thereby manipulate the spinal fixation element 500 as desired.

Once the spinal fixation element 500 has been inserted and positioned as desired, the spinal fixation element 500 may then be connected to the implants 100a and 100b to fix the position of the spinal fixation element 500. To achieve this, closure mechanisms are used.

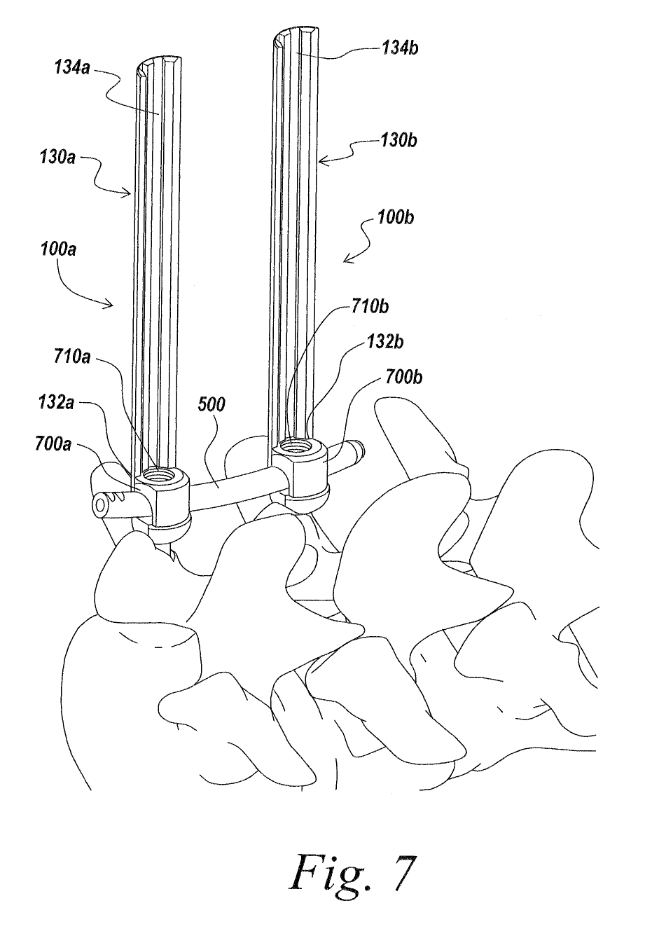

An example using closure mechanisms can be seen in FIG. 7. In this embodiment, a first closure mechanism 700a is inserted using the guide tab 130a of the first implant 100a. A second closure mechanism 700b is also inserted using the guide tab 130b of the second implant 100b.

If the implant 100a and 100b where inserted percutaneously, as describe above, the closure mechanisms 700a and 700b may be inserted through the same incisions used to insert the implant 100a and 100b. In certain embodiments, the closure mechanism 700a and 700b are configured to engage the surface configurations 134a and 134b of the guide tabs 130a and 130b. For example, the closure mechanisms 700a and 700b may be keyed to ride in rails provide by the surface configurations 134a and 134b. Thus, the closure mechanisms 700a and 700b may be slide along the guide tabs 130a and 130b from outside the patient to the connector body 120 inside the body to capture the spinal fixation element 500.

Once the spinal fixation element 500 has been captured by the closure mechanisms 700a and 700b, the closure mechanisms 700a and 700b may be secured using locking mechanisms 710a and 710b. In the example of FIG. 7, the locking mechanisms 710a and 710b are set screws. In this example the set screws 710a and 710b have been preset into the closure mechanisms 700a and 700b. Thus, the set screws 710a and 710b just require tightening using a driver (not shown) to secure the spinal fixation element 500.

Once the spinal fixation element 500 has been connected to the implants 200a and 200b using the closure mechanisms 700a and 700b (steps 250 and 260), the guide tabs 130a and 130b of the implants 100a and 100b may be detached and removed In the case of the embodiments set forth above, this involves detaching the first guide tab 130a from the first implant 100a (step 270) and detaching the second guide tab 130b from the second implant 100b (step 280). As discussed previously, the guide tabs 130a and 130b of the implants 100a and 100b may include fragile or weakened or otherwise breakaway features 132a and 132b allowing for easier detachment and removal.

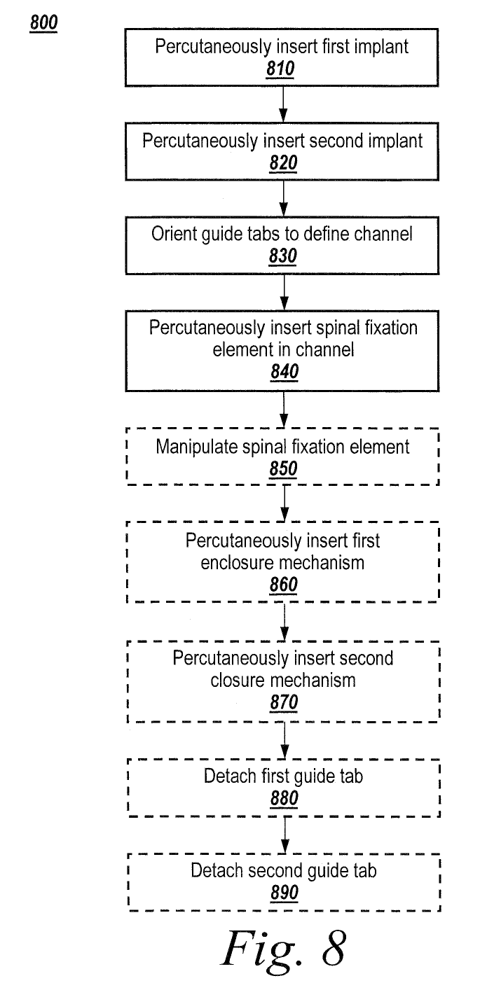

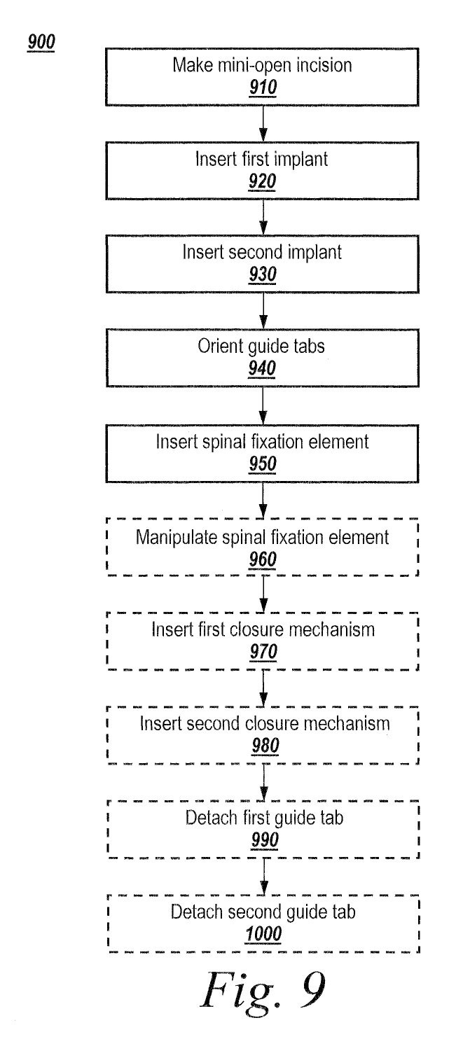

While the exemplary embodiment of FIG. 2 depicts a generic method of the present invention, it should be understood that the methodology may be adjusted to reflect particular insertion techniques. Some examples of other embodiments of methodologies can be seen in FIGS. 8 and 9.

FIG. 8 depicts a flow diagram 800 of one exemplary embodiment wherein the implants and spinal fixation element are percutaneously inserted using the techniques described above with the implants orientated as seen in FIGS. 6A and 6B. A first implant is percutaneously inserted into a first vertebra of the patient's spine (Step 810). A second implant may then be percutaneously inserted into a second vertebra of the patient's spine (Step 820). The techniques for percutaneous insertion are discussed above in reference to FIGS. 3A-D.

The guide tabs of the first and second implants may then be orientated to define a channel along the patient's spine for receiving the spinal fixation element (step 830). This orientation is described above in reference to FIGS. 6A and 6B. Additional implants may also be inserted and orientated as desired.

A spinal fixation element, such as a rod, may then be percutaneously inserted within the channel defined by the guide tabs of the first and second implants to locate the spinal fixation element along the patient's spine (Step 840). This was also discussed above in regard to FIGS. 6A and 6B.

As with the method of FIG. 1, in certain embodiments, the method of FIG. 8 may include additional steps. After insertion, the spinal fixation element may be further manipulated using the guide tabs of the first and second implants (step 850). Once the implants and spinal fixation element have been inserted (and manipulated), closure mechanisms may be inserted to connect the spinal fixation element to the implants. In the example of FIG. 8, a first closure mechanism may be percutaneously inserted to connect the spinal fixation element to the first implant (Step 860). A second closure mechanism may also be percutaneously inserted to connect the spinal fixation element to the second implant (Step 870). It should be understood that the techniques discussed above in regard to FIG. 7 may performed on implants having the orientation shown in FIGS. 6A and 6B. Additional closure mechanism may further be inserted to connect the spinal fixation element to any additionally inserted implants as desired.

In other embodiments, once the spinal fixation element has been connected to the implants, the guide tabs may be removed. In the example of FIG. 8, the guide tab the first implant may be detached from the first implant (Step 880) and the guide tab of the second implant may be detached from the second implant (Step 890). Additional guide tabs may further detached from any additionally inserted implants.

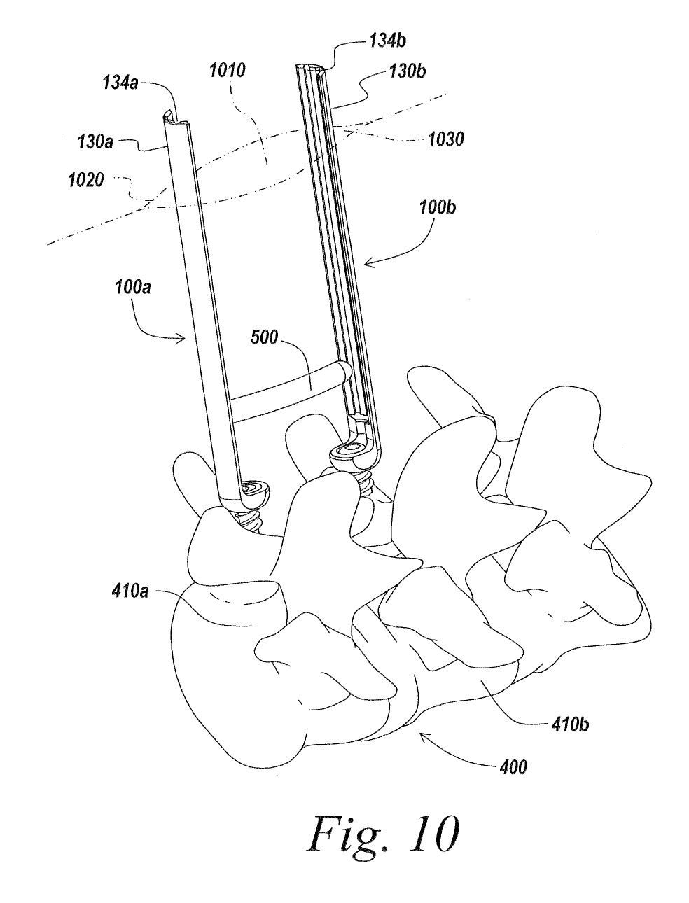

While the previous examples dealt primarily with percutaneous insertion, it should be understood the method may be practiced using other types of insertion techniques may be used. One possible alternative exemplary embodiment can be seen in FIGS. 9 and 10.

FIG. 9 depicts a flow diagram 900 of one exemplary embodiment wherein the implants and spinal fixation element are inserted through a mini-open incision. First a mini-open incision is made along the patient's spine (step 910). A first implant may then be inserted through the mini-open incision into a first vertebra (step 920). A second implant may then be inserted through the mini-open incision into a second vertebra (step 930). The guide tabs of the first and second implants may then be orientated to receive and guide the spinal fixation element (step 940). Once the guide tabs are properly orientated, the spinal fixation element may inserted through the mini-incision along the guide tabs of the first and second implants (step 950) Each of these steps will be described in more detail below.

In certain embodiments, the method may further include additional steps. After insertion, the spinal fixation element may be further manipulated using the guide tabs of the first and second implants (step 960). Once the implants and spinal fixation element have been inserted (and manipulated), closure mechanisms may be inserted to connect the spinal fixation element to the implants. In the example of FIG. 9, a first closure mechanism may be inserted to connect the spinal fixation element to the first implant (Step 970). A second closure mechanism may also be inserted to connect the spinal fixation element to the second implant (Step 980). Additional closure mechanism may further be inserted to connect the spinal fixation element to any additionally inserted implants as desired. Each of these steps will be discussed in further detail below.

In other embodiments, once the spinal fixation element has been connected to the implants, the guide tabs may be removed. In the example of FIG. 9, the guide tab the first implant may be detached from the first implant (Step 990) and the guide tab of the second implant may be detached from the second implant (Step 1000). Additional guide tabs may further detached from any additionally inserted implants. Each of these steps will be discussed in further detail below.

A mini-incision is a minimally invasive surgical (MIS) technique in which a small incision on the patient along the spine is used to insert the implants and spinal fixation element. The incision is larger than used for percutaneous insertion but smaller than used in traditional techniques. In traditional techniques the incision may be span the entire of the patient's spine while a mini incision may just span the distance between two vertebrae on the patient's spine. An example of this can be seen in FIG. 10.

In FIG. 10, a mini incision 1010 is made spanning between a first 410a and second 410b vertebra of the patient's spine 400. (step 910). A first implant 100a has been inserted through the mini-incision 1010 into the first vertebra 410a and a second implant 200b has been inserted through the mini-incision 1010 into the second vertebra 410b (steps 920 and 930). In certain embodiments, the first implant 100a is located proximate to a first end 1020 of the mini-incision 1010 while the second implant 100b is located proximate to a second end 1030 of the mini-incision 1010 such that the guide tabs 130a and 130b of the implants 100a and 100b extend outside of the patient and help define the opening of the mini-incision 1010.

In the example of FIG. 10, the first guide tab 130a and second guide tab 130b have been orientated (step 940) to be at opposite ends of an inserted spinal fixation element 500. The spinal fixation element 500 may then be inserted through the mini-incision 1010 between the guide tabs 130a and 130b (step 950) as shown in FIG. 10. Here, the partial cannula shape and surface configuration 134a and 134b of the guide tabs 130a and 130b serve to engage and guide the spinal fixation element into location along the patient's spine. It should be understood however, that FIG. 10 depicts one possible orientation. The guide tabs may also be orientated as seen in FIGS. 4-7 or any other orientation when using a mini-incision technique.

The steps of manipulation (step 960), inserting closure mechanisms (steps 970 and 980), and removing the guide tabs (steps 990 and 1000) may then be performed as discussed above. It will be apparent to one skilled in the art that the techniques and device discussed above for these steps can be modified for the particular orientation and insertion technique shown in FIG. 10 without departing from the spirit and scope of the invention.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

While the methods and instruments of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

1. A method for inserting a spinal fixation element along a patient's spine, the method comprising:
inserting a first implant having a guide tab into a first vertebra;
inserting a second implant having a guide tab into a second vertebra; and
inserting the spinal fixation element using the guide tabs of the first and second implants to locate the spinal fixation element along the patient's spine.

2. The method of claim 1, wherein the first and second implants comprise polyaxial implants.

3. The method of claim 1, wherein the guide tabs of the first and second implant extend through the patients skin after the first and second implants have been inserted.

4. The method of claim 1, wherein the first and second implants comprise:
a bone anchor having a proximal head and a distal shaft extending along a longitudinal axis configured to engage bone;
a connector body configured to engage the proximal head of bone anchor and connector body a spinal fixation element; and
a detachable guide tab extending from the connector body opposite and offset from the distal shaft of the bone anchor.

5. The method of claim 1, further comprising:
inserting a first closure mechanism to connect the inserted spinal fixation element to the first inserted implant; and
inserting a second closure mechanism to connect the inserted spinal fixation element to the second inserted implant.

6. The method of claim 5, wherein the first closure mechanism is inserted using the guide tab of the first implant and the second closure mechanism is inserted using the guide tab of the second implant.

7. The method of claim 5, further comprising:
detaching the guide tab from the first inserted implant after the spinal fixation element has been connected to the first implant with the first closure mechanism; and
detaching the guide tab from the second inserted implant after the spinal fixation element has been connected to the second implant with the second closure mechanism

8. The method of claim 1, wherein the first implant, second implant, and spinal fixation element are inserted percutaneously.

9. The method of claim 8, wherein the spinal fixation element is inserted through the same incision as the first or second implant.

10. The method of claim 8, wherein the spinal fixation element is inserted through a separate incision than the first or second implant.

11. The method of claim 1, wherein the guide tabs of the first and second implants are orientated to be at opposite sides of the spinal fixation element to locate the spinal fixation element along the patients spine.

12. The method of claim 1, wherein the first implant, second implant, and spinal fixation element are inserted using a mini-open procedure.

13. The method of claim 12, wherein the guide tabs of the first and second implants are orientated to be at opposite ends of the spinal fixation element to locate the spinal fixation element along the patients spine.

14. The method of claim 1, wherein the spinal fixation element is inserted before first and second implants and then located along the patients spine after the first and second implant have been inserted.

15. The method of claim 14, wherein the guide tabs of the first and second implants are orientated to be on the same side of the spinal fixation element to locate the spinal fixation element along the patients spine.

16. The method of claim 1, further comprising manipulating the position of spinal fixation element using the guide tabs of the first and second implant.

17. A method for inserting a spinal fixation element along a patient's spine, the method comprising:
inserting, percutaneously, a first implant having a guide tab into a first vertebra;
inserting, percutaneously, a second implant having a guide tab into a second vertebra;
orientating the guide tabs of the first and second implants to define a channel along the patient's spine for receiving the spinal fixation element;
inserting, percutaneously, the spinal fixation element within the channel defined by the guide tabs of the first and second implants to locate the spinal fixation element along the patient's spine.

18. The method of claim 17, wherein the first and second implants comprise:
a bone anchor having a proximal head and a distal shaft extending along a longitudinal axis configured to engage bone;
a connector body configured to engage the proximal head of bone anchor and connector body a spinal fixation element; and
a detachable guide tab extending from the connector body opposite and offset from the distal shaft of the bone anchor.

19. The method of claim 17, further comprising:
inserting, percutaneously, a first closure mechanism to connect the inserted spinal fixation element to the first inserted implant; and
inserting, percutaneously, a second closure mechanism to connect the inserted spinal fixation element to the second inserted implant.

20. The method of claim 19, wherein the first closure mechanism is inserted using the guide tab of the first implant and the second closure mechanism is inserted using the guide tab of the second implant.

21. The method of claim 19, further comprising:
detaching the guide tab from the first inserted implant after the spinal fixation element has been connected to the first implant with the first closure mechanism; and
detaching the guide tab from the second inserted implant after the spinal fixation element has been connected to the second implant with the second closure mechanism.

22. A method for inserting a spinal fixation element along a patient's spine, the method comprising:
making a mini-open incision along the patient's spine;
inserting, through the mini-open incision, a first implant having a guide tab into a first vertebra proximal to a first end of the mini-open incision;
inserting, though the mini-open incision, a second implant having a guide tab into a second vertebra proximal to a second end of the mini-open incision;
orientating the guide tabs of the first and second implants to receive and guide first and second ends of the spinal fixation element; and
inserting, though the mini-open incision, the spinal fixation element between the guide tabs of the first and second implants,
wherein the guide tabs of the first and second implants receive the first and second ends of the spinal fixation element and guide the insertion of the spinal fixation element along the patient's spine.

23. The method of claim 22, wherein the first and second implants comprise:
a bone anchor having a proximal head and a distal shaft extending along a longitudinal axis configured to engage bone;
a connector body configured to engage the proximal head of bone anchor and connector body a spinal fixation element; and
a detachable guide tab extending from the connector body opposite and offset from the distal shaft of the bone anchor.

24. The method of claim 22, further comprising:
inserting a first closure mechanism to connect the inserted spinal fixation element to the first inserted implant; and
inserting a second closure mechanism to connect the inserted spinal fixation element to the second inserted implant.

25. The method of claim 24, wherein the first closure mechanism is inserted using the guide tab of the first implant and the second closure mechanism is inserted using the guide tab of the second implant.

26. The method of claim 24, further comprising:
detaching the guide tab from the first inserted implant after the spinal fixation element has been connected to the first implant with the first closure mechanism; and
detaching the guide tab from the second inserted implant after the spinal fixation element has been connected to the second implant with the second closure mechanism
